(19) United States
(12) Patent Application Publication
(10) Pub. No.: US 2013/0204125 A1
(43) Pub. Date: Aug. 8, 2013

(54) INFLATION APPARATUS WITH PRESSURE RELIEF, RELATED SYSTEMS, METHODS AND KITS

(71) Applicants: Narissa Y. Chang, Somerville, MA (US); James W. Moriarty, JR., Georgetown, MA (US); George J. Purtell, Westford, MA (US); Mathew Puthenvila, Waltham, MA (US)

(72) Inventors: Narissa Y. Chang, Somerville, MA (US); James W. Moriarty, JR., Georgetown, MA (US); George J. Purtell, Westford, MA (US); Mathew Puthenvila, Waltham, MA (US)

(73) Assignee: NinePoint Medical, Inc., Cambridge, MA (US)

(21) Appl. No.: 13/365,445

(22) Filed: Feb. 3, 2012

(51) Int. Cl. A61M 25/10; A61B 6/00

(52) U.S. Cl. 600/425; 604/99.02; 604/98.01

(57) ABSTRACT

Systems, devices, methods and kits for an inflation system with pressure relief are provided. The system includes an inflatable member, a first shaft connected to the inflatable member, an imaging device extending into said cavity of the inflatable member, a second shaft configured to contain the imaging device, the second shaft having a closed end approximate to the imaging assembly and a open end approximate to the imaging system, the second shaft defining a cavity along a longitudinal axis thereof and configured to be positioned within the cavity of the first shaft; the first shaft and the second shaft defining a channel therebetween in communication with the cavity of the inflatable member; an inflator connected to the first shaft and in communication with the channel for inflating the inflatable member; and a relief valve in communication with the channel and positioned between the inflatable member and the inflator.

### TECHNICAL FIELD

The present disclosure generally relates to medical devices, systems and methods for that include an inflatable member in biomedical and other medical and non-medical applications, and in particular to apparatuses, systems, methods and kits for preventing over inflation of an inflatable member.

### BACKGROUND

Various types of inflatable members are used during medical procedures to expand an internal cavity of a patient in order to perform a medical procedure.

One type of inflatable member is the balloon catheter. In general, balloon catheters can exist in a deflated state and an inflated state; intermediate states are also available. In use, the balloon catheter in its deflated state is inserted into a cavity of a patient. After positioning within the patient, the balloon catheter is inflated via any of various means using various inflation media, for example, using a syringe to inject a liquid mass into the balloon or using an inflation bulb to provide air into the balloon. Some systems utilize a pressure gauge to monitor the pressure to prevent over pressurization of the balloon.

In particular, in some medical procedures an imaging device is used to image an internal cavity of a patient. In order to capture clear images of the cavity tissue, the imaging device can be positioned within a balloon catheter that can be inserted into the cavity. The balloon is then inflated to provide clear access to the imaging device of the system. In these balloon catheter systems, the balloon catheter and most components connected thereto require disposal due to being in contact with the patient.

In some instances, if an operator is not properly monitoring the pressure gauge, the balloon may be inflated to an over inflated or over pressurized state. This over pressurization of the balloon can cause damage to or even rupturing of the balloon, or even worse can cause damage to the surround tissue within the cavity of the patient. Also if the balloon is underinflated, the imaging device may not be able to properly capture and image of the surrounding tissue. This disclosure describes an improvement over these prior art technologies.

### SUMMARY

Accordingly, an inflation apparatus with pressure relief is provided that includes an inflatable member having a proximal end and a distal end and defining a deflated state, an inflated state, and a cavity therein; a first shaft having a first end connected to the proximal end of the inflatable member and defining a cavity along a longitudinal axis thereof; an imaging device having an imaging assembly at a distal end thereof and extending into said cavity of said inflatable member and connectable to an imaging system at a proximal end thereof; a second shaft configured to contain said imaging device, said second shaft having a closed end approximate to the imaging assembly and a open end approximate to the imaging system, said second shaft defining a cavity along a longitudinal axis thereof and configured to be positioned within said cavity of said first shaft; said first shaft and said second shaft defining a channel therebetween in communication with said cavity of the inflatable member; an inflator connected to said first shaft and in communication with said channel for inflating said inflatable member; and a relief valve in communication with said channel and positioned between said inflatable member and said inflator.

In one embodiment, an inflation apparatus with pressure relief includes an inflatable member having a deflated state and an inflated state, and defining a cavity therein; a first shaft defining a cavity therein and having a proximal end and a distal end, said distal end connected to said inflatable member; a second shaft defining a cavity therein and having a closed end and an open end, said second shaft disposed within said first shaft such that said closed end is disposed within said inflatable member, said first shaft and said second shaft defining a channel therebetween in communication with the cavity of the inflatable member; an inflator in communication with the channel configured to inflate the inflatable member; a relief valve in communication with the channel to prevent over pressurization if the inflatable member; and a pass-through component configured to maintain an isolation of the cavity of the second shaft from said channel and permit communication between said inflator and said channel.

In one embodiment, an inflation kit with pressure relief includes more than one air supply for supplying air through a pathway to an inflatable member; a valve connected in the pathway to control the flow of the inflatable member; a pressure gauge connected in the pathway for monitoring the pressure of the inflatable member; and a pressure relief valve connected in the pathway for venting the pressure at a preset pressure.

In one embodiment a method for testing an inflation kit includes receiving an inflation kit; attaching a test valve to the pathway and configured to seal the pathway; closing test valve to seal the pathway; opening the valve to increase pressure in the pathway; monitoring pressure gauge; closing the valve upon reaching a preset pressure; determining is the pressure is maintained for a preset period of time; after the preset period of time, opening the valve to again increase the pressure in the pathway; monitoring the pressure gauge; determining if the relief valve opens; identifying on the pressure gauge the pressure at which the relief valve opens.

### BRIEF DESCRIPTION OF THE DRAWINGS

The present disclosure will become more readily apparent from the specific description accompanied by the following drawings, in which:

FIG. 1 is a schematic diagram of an inflation system with pressure relief in accordance with the principles of the present disclosure;

FIG. 2 is a partial front view of the inflation system of FIG. 1;

FIG. 3 is a cross sectional view of the system of FIG. 1 at a balloon end thereof; and

FIG. 4 is a cross sectional view of an upper end of the system of FIG. 1.

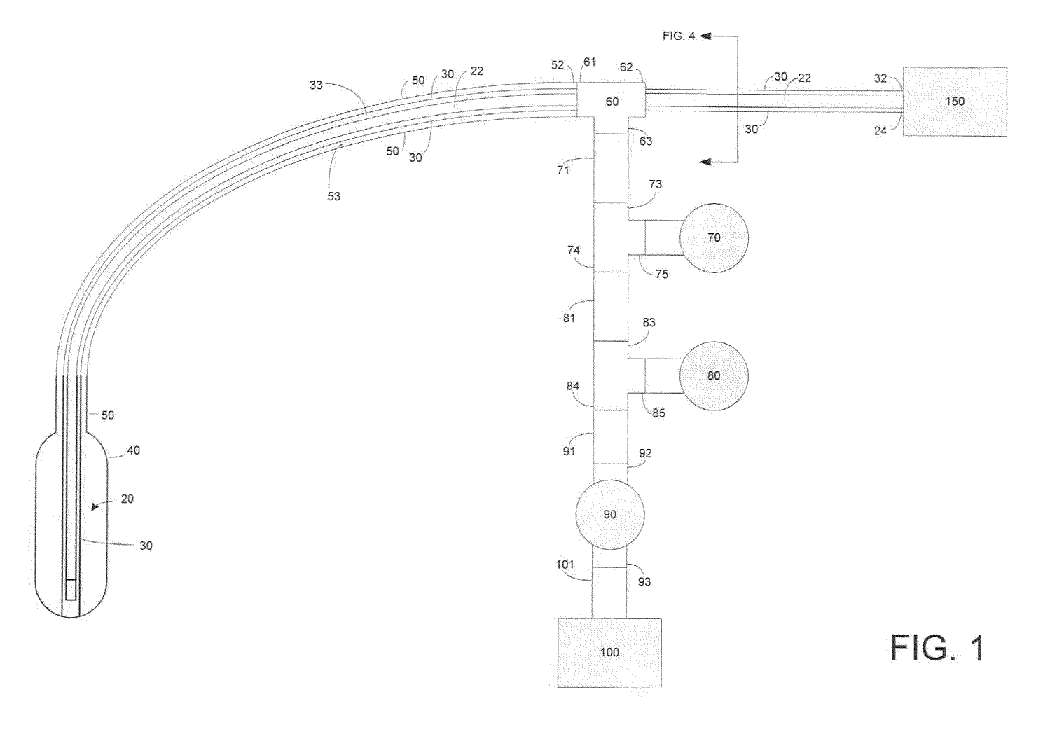

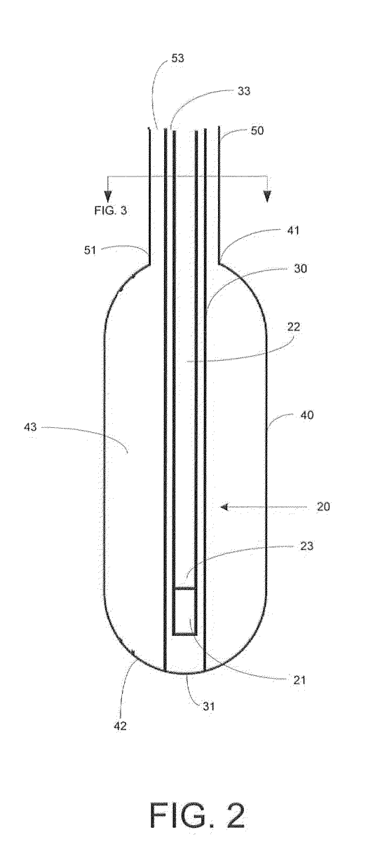

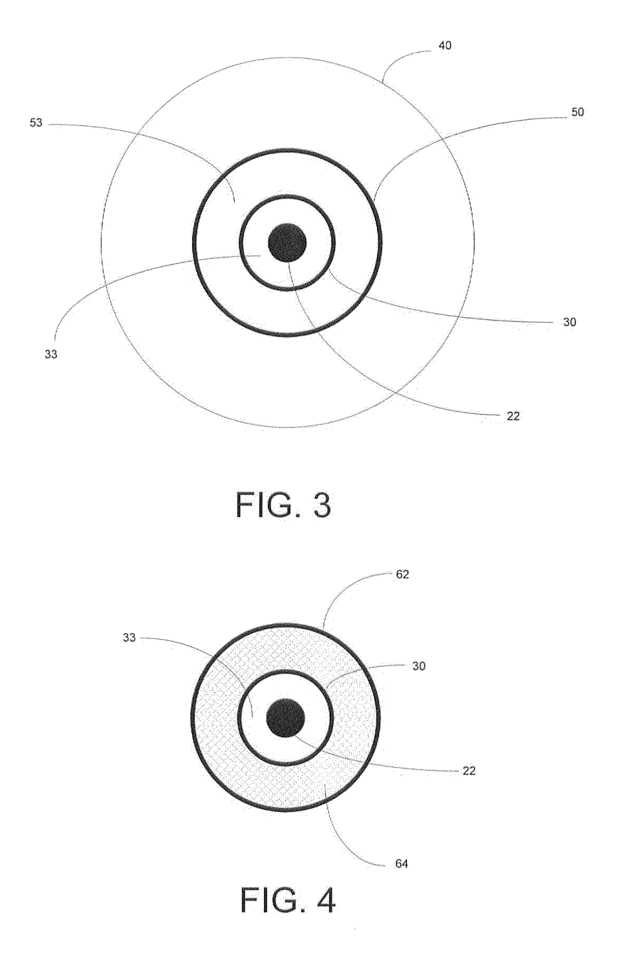

Like reference numerals indicate similar parts throughout the figures.

### DETAILED DESCRIPTION

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure.

The present disclosure is described herein in connection with an imaging system. It is understood that the present disclosure is applicable to any systems that include an inflatable member, the pressure of which is to be monitored and controlled.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

Reference will now be made in detail to the exemplary embodiments of the present disclosure, which are illustrated in the accompanying figures.

System 10 includes an imaging device 20, e.g. an optical coherence tomography (OCT) imaging device. Although the present disclosure is described using an OCT imaging device, other imaging devices are contemplated. For example, imaging device can include a visual light camera, an ultrasound imaging device or other imaging devices. OCT imaging device 20 includes an imaging assembly 21 comprising one or more components commonly found in rotating and/or translating imaging devices. These components can include mirrors, lenses, filters, prisms and combinations thereof; other components are contemplated. OCT imaging device 20 is connected to a distal end 23 of an inner member 22. When used in connection with OCT imaging device 20, inner member 22 can include a fiber optic cable configured to transmit light energy. A proximal end 24 of inner member 22 is connected to one or more imaging systems 150, e.g. an OCT visualization system.

OCT imaging device 20 is contained within an inner shaft 30 having a distal end 31 and a proximal end 32. Inner shaft 30 is sealed at distal end 31 and can attach to imaging system 150 at proximal end 32. Inner shaft 30 provides a working environment for OCT imaging device 20 to freely rotate and/or translate within. Inner channel 33 is defined between inner member 22 and inner shaft 30. Inner shaft 30 can be rigid or flexible depending on the system requirements.

Distal end 31 of inner shaft 30 containing OCT imaging device 20 is contained within an inflatable member 40, e.g. a balloon, having a proximal end 41 and a distal end 42. Balloon 40 defines an inner cavity 43. Balloon 40 can be manufactured from various compliant and/or non-compliant materials, for example, latex and/or polyethylene terephthalate (PET), polyurethane, nylon or polyether block amide. Other materials are contemplated. Whichever material is used, balloon 40 is designed to transition between a deflated state and an inflated state; intermediate states are contemplated. Balloon 40 is shown in an inflated state.

Proximal end 41 of balloon 40 is connected to a distal end 51 of an outer shaft 50. Outer shaft 50 can be rigid or flexible depending on the system requirements. Outer shaft 50 is configured to slidingly receive inner shaft 30 and OCT imaging device 20. Distal end 31 of inner shaft 30 can be attached to distal end 42 of balloon 40. An outer channel 53 is defined between inner shaft 30 and outer shaft 50. Outer channel 53 is in communication with cavity 43 of balloon 40. Outer channel 53 is used to deliver or remove air to/from cavity 43 to inflate or deflate balloon 40. Inner channel 33 is sealed from and does not communicate with outer channel 53.

A proximal end 52 of outer shaft 50 is connected to a first end 61 of a branch tee 60. Although a branch tee is described herein, other fillings are contemplated, for example, a heal tee or Y shaped fitting can also be used. Branch tee 60 is designed to allow inner shaft 30 and inner member 22 to pass therethrough but retain the seal between inner channel 33 and outer channel 53. Inner shaft 30 and inner member 22 extend from a second end 62 of branch tee 60 to connect to imaging system 150. As shown in FIG. 4, inner shaft 30 and inner member 22 extend from second end 66 of branch tee 60. A space 64 between an outer surface of inner shaft 30 and an inner surface of second end 62 is sealed to seal channel 53 from the outside environment. Seal of space 64 can be a sealant or can be monolithically formed with tee 60 to tightly seal around inner shaft 30. As another example, branch tee 60 can be molded over the inner shaft 30 and thermally bonded thereto to seal around it.

The bull 63 of branch tee 60 is connected to a first end 73 of a branch tee 72 via tube 71. A second end 74 of branch tee 72 is connected to a first end 83 of a branch tee 82 via tube 81. A second end 84 of branch tee 82 is connected to an outlet 92 of a valve 90 via tube 91. An inlet 93 of valve 90 is connected to air supply 100 via tube 101. Tubes 71, 81, 91 and/or 101 can be rigid or flexible depending on system requirements. Although tubes are described as connecting various components (e.g. tees 72 and 82), direct connections between the components are contemplated. In addition, the orientation of the components can vary depending on system configuration.

Air supply 100 can include mechanical, electromechanical or pressurized air supplies. For example, air supply can include an inflation bulb, a syringe, an electric pump or an air tank containing pressurized air. Other air supplies are contemplated. In addition, as stated above, the present disclosure is not limited to using air to inflate the balloon. For example other gases such as nitrogen or helium or liquids such as saline or contrast media are contemplated.

A relief valve 70 is connected to bull 75 of tee 72. Relief valve 70 is designed to prevent an over pressuring of balloon 40. For example, in a system wherein an esophagus of a patient is to be imaged, balloon 40, in a deflated state and containing OCT imaging device 20, is inserted into the patient. Before imaging can commence, balloon 40 requires inflation. A PET balloon for this application may require a pressure of 5 pounds per square inch (psi) as a nominal pressure to properly inflate. Such a PET balloon 40 may have a pressure tolerance rating of +5 psi. As such, a relief valve 70 designed to release at 8 psi +/−2 can be used to. maintain balloon 40 within its tolerance ranges. Based on the specifications of the balloon 40, differing pressure valves can be used.

A pressure gauge 80 is connected to bull 85 of tee 82. Pressure gauge 80 is used to monitor the pressure in the balloon 40 as cavity 43 is connected to pressure gauge 80 through channel 53.

The present disclosure describes a inflation apparatus with pressure relief that can be reused. That is, the components from branch tee 60 through air supply 100 are tangential to the path of balloon 40 and shaft 50, and thus the patient, and therefore can be reused and remain non-sterile.

In use and operation, balloon 40, in a deflated state and containing imaging device 20, is inserted into a cavity of a patient to be imaged. Once at the desired position, air pressure created by air supply 100 is allowed to enter the system 10 by the opening of valve 90. As the air pressure increases, balloon 40 transitions from its deflated state to its inflated state. During this process, pressure gauge 80 can be monitored to monitor the increasing pressure in the system 10. In normal operation this process continues until a desired pressure, e.g. 5 psi, is reached, at which time valve 90 would be closed to prevent over pressurization. In the event the monitoring of pressure gauge 80 is interrupted, thus allowing the air pressure in the system to continue to increase, relief valve will open at its set pressure, e.g. 8 psi +/−2, to prevent damage to the system 10 or the patient.

Due to the design of the system 10, the system 10 can maintain a required balloon 40 pressure and allow the operation of the OCT imaging device 20 to translate and/or rotate the image 21 within the patient.

An inflation kit is also contemplated. The kit can include more than one air supply 100, for example, an inflation bulb and a syringe. Also included in the kit are relief valve 70, pressure gauge 80 and valve 90 connected via tubing as described herein. The kit comes ready to connect to branch tee 60.

The inflation apparatus with pressure relief can also be subject to a pressure testing procedure. A sub-system of components 71 through 100 are assembled as described. A test valve (not shown) is attached to end of tubing 71, replacing branch tee 60. With the test valve in a closed position and valve 90 in an opened position, pressure is applied to the sub-system via air supply 100. Pressure gauge 80 is monitored until a preset pressure is obtained, e.g. 5 psi. This pressure is maintained, i.e. valve 90 is closed, for a preset time period, e.g. 30 seconds. After the preset time period has elapsed, the pressure is again increased by opening valve 90. The pressure is monitored via pressure gauge 80 until relief valve 70 opens at its preset pressure, e.g. 8 psi. A calibrated and tested secondary relief valve can be incorporated into the sub-system to prevent damage to the sub-system should relief valve 70 fail to operate properly. In addition, a calibrated and tested secondary pressure gauge can be incorporated into the sub-system to accurately determine if the relief valve opens within its specified range and determine if pressure gauge 80 is accurate.

The present disclosure has been described herein in connection with an imaging system including an OCT imaging device contained within a balloon. It is understood that the present disclosure is applicable to any systems that include an inflatable member, the pressure of which is to be monitored, with or without imaging devices as disclosed herein. For example, the present disclosure is applicable to systems for performing procedures such as angioplasty. Other applications are contemplated.

Where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims.

What is claimed is:

1. An inflation system with pressure relief, comprising:
an inflatable member having a proximal end and a distal end and defining a deflated state, an inflated state, and a cavity therein;
a first shaft having a first end connected to the proximal end of the inflatable member and defining a cavity along a longitudinal axis thereof;
an imaging device having an imaging assembly at a distal end thereof and extending into said cavity of said inflatable member and connectable to an imaging system at a proximal end thereof;
a second shaft configured to contain said imaging device, said second shaft having a closed end approximate to the imaging assembly and a open end approximate to the imaging system, said second shaft defining a cavity along a longitudinal axis thereof and configured to be positioned within said cavity of said first shaft;
said first shaft and said second shaft defining a channel therebetween in communication with said cavity of the inflatable member;
an inflator connected to said first shaft and in communication with said channel for inflating said inflatable member;
a relief valve in communication with said channel and positioned between said inflatable member and said inflator.

2. The system of claim 1, further comprising a pressure gauge in communication with said channel.

3. The system of claim 1, further comprising a shutoff valve in communication with said channel and positioned between said inflator and said relief valve.

4. The system of claim 1, wherein said cavity of said second shaft is isolated from said channel.

5. The system of claim 4, further comprising a pass-through component configured to maintain the isolation of the cavity of the second shaft from said channel, permit pass through of said imaging device, and permit continued communication between said inflator and said channel.

6. The system of claim 1, wherein the inflatable member is a medical balloon.

7. The system of claim 1, wherein said imaging device in an optical coherence tomography (OCT) imaging device and the imaging system is an OCT imaging system.

8. The system of claim 1, wherein said inflator supplies one of a gas or a liquid into said channel.

9. The system of claim 1, wherein said inflator is one of a mechanical, electromechanical or pressurized supply of air.

10. An inflation system with pressure relief, comprising:
an inflatable member having a deflated state and an inflated state, and defining a cavity therein;
a first shaft defining a cavity therein and having a proximal end and a distal end, said distal end connected to said inflatable member;
a second shaft defining a cavity therein and having a closed end and an open end, said second shaft disposed within said first shaft such that said closed end is disposed within said inflatable member, said first shaft and said second shaft defining a channel therebetween in communication with the cavity of the inflatable member;
an inflator in communication with the channel configured to inflate the inflatable member;
a relief valve in communication with the channel to prevent over pressurization if the inflatable member; and
a pass-through component configured to maintain an isolation of the cavity of the second shaft from said channel and permit communication between said inflator and said channel.

11. The system of claim 10, further comprising a pressure gauge in communication with said channel.

12. The system of claim 10, further comprising a shutoff valve in communication with said channel and positioned between said inflator and said relief valve.

13. The system of claim 10, wherein the inflatable member is a medical balloon.

14. The system of claim 10, wherein said imaging device in an optical coherence tomography (OCT) imaging device and the imaging system is an OCT imaging system.

15. The system of claim 10, wherein said inflator supplies one of a gas or a liquid into said channel.

16. The system of claim 10, wherein said inflator is one of a mechanical, electromechanical or pressurized supply of air.

17. An inflation kit with pressure relief: comprising:
more than one air supply for supplying air through a pathway to an inflatable member;
a valve connected in the pathway to control the flow of the inflatable member;
a pressure gauge connected in the pathway for monitoring the pressure of the inflatable member; and
a pressure relief valve connected in the pathway for venting the pressure at a preset pressure.

18. The kit of claim 17, wherein the more than one air supply includes an inflation bulb and a syringe.

19. The kit of claim 17, wherein the valve, the pressure gauge and the relief valve are connected via tubing.

20. A method for testing an inflation kit of claim 17, comprising the steps of:
receiving an inflation kit;
attaching a test valve to the pathway and configured to seal the pathway;
closing test valve to seal the pathway;
opening the valve to increase pressure in the pathway;
monitoring pressure gauge;
closing the valve upon reaching a preset pressure;
determining is the pressure is maintained for a preset period of time;
after the preset period of time, opening the valve to again increase the pressure in the pathway;
monitoring the pressure gauge;
determining if the relief valve opens;
identifying on the pressure gauge the pressure at which the relief valve opens.
